(19) United States
(12) Patent Application Publication
(10) Pub. No.: US 2018/0168679 A1
(43) Pub. Date: Jun. 21, 2018

(54) SURGICAL SNARE INSTRUMENT

(71) Applicants: Hyung Jong OH, Gwangju (KR); GNST CO., LTD., Gwangju (KR)

(72) Inventors: Hyung Jong OH, Gwangju (KR); Jae Seung CHO, Gwangju (KR); Nam In JEONG, Gwangju (KR)

(21) Appl. No.: 15/381,196

(22) Filed: Dec. 16, 2016

(51) Int. Cl. A61B 17/3205; A61B 17/12; A61B 17/32

(52) U.S. Cl. CPC A61B 17/32056; A61B 2017/00367; A61B 17/320016; A61B 17/12013

(57) ABSTRACT

The present disclosure relates to a surgical snare instrument that includes a tube having a hollow, an excising unit having a wire installed to be slidable along the hollow of the tube and a loop part installed at an end of the wire and inserted into one end of the tube to be tightened to excise a polyp, an advancing-and-retracting unit installed at the other end of the tube to make the wire advance or retract along the hollow of the tube, and a rotating unit installed adjacent to the one end of the tube and configured to rotate the loop part when the wire moves with respect to the tube.

### BACKGROUND

### Field of the Invention

The present disclosure relates to a surgical snare instrument, and more particularly, to a surgical snare instrument used in surgery for removing a part of an organ inside a body.

### Discussion of Related Art

Generally, colon polyps refer to lumps, caused by abnormal growth of intestinal mucosa, protruding inward from inside an intestine. In such case, polypectomy is performed.

Polypectomy refers to completely excising polyps having a predetermined size or larger using a polypectomy tool. Normally, polyps having sizes of 5 mm or larger are removed.

A snare instrument for an endoscope is disclosed in Korean Unexamined Patent Application Publication No. 10-2016-0035930. The disclosed snare instrument includes a manipulation part, a driving cord configured to be advanced and retracted by operation of the manipulation part, an electrically insulating tube through which the driving cord passes, and an electrically conductive loop wire connected to the front end of the driving cord to be advanced and retracted with the driving cord and installed so that at least a portion thereof is exposed to outside of the front end of the electrically insulating tube and then introduced into the electrically insulating tube after being retracted.

The snare instrument performs polypectomy on a part where polyps to be removed are formed, using an endoscope and the electrically conductive loop wire. Here, the electrically conductive loop wire has to be rotated in a direction in which the polyps are formed. However, there is a problem in that it is impossible to only rotate the electrically conductive loop wire, and the whole instrument has to be rotated. This problem causes an uncomfortable posture for an operator and difficulties in accurately setting direction of the loop wire.

### RELATED ART DOCUMENT

### Patent Document

(Patent document 1) Korean Unexamined Patent Application Publication No. 10-2016-0035930: Snare instrument for endoscope

### SUMMARY OF THE INVENTION

The present disclosure is directed to providing a surgical snare instrument in which a loop part of an excising unit is easily rotatable in a direction of polyps to be excised.

To achieve the above objectives, a surgical snare instrument according to the present disclosure includes a tube having a hollow, an excising unit having a wire installed to be slidable along the hollow of the tube and a loop part installed at an end of the wire and inserted into one end of the tube to be tightened to excise a polyp, an advancing-and-retracting unit installed at the other end of the tube to make the wire advance or retract along the hollow of the tube, and a rotating unit installed adjacent to the one end of the tube and configured to rotate the loop part when the wire moves with respect to the tube.

According to the present disclosure, the advancing-and-retracting unit may include a body portion including a first insertion portion into which a finger is insertable and an extension frame configured to extend from the first insertion portion and have a guide part formed in a longitudinal direction and an end to which the tube is coupled; and a handle portion including second and third insertion portions into which a finger is insertable and a moving frame configured to connect the second and third insertion portions to each other, have an insertion hole into which the extension frame is inserted to be movable in the longitudinal direction of the extension frame, and have the wire of the excising unit fixed thereto.

The rotating unit may include a contact protrusion configured to protrude in a radial direction from a wire adjacent to the loop part. The hollow formed in the tube by an end of the tube helically twisted so that the contact protrusion is guided and the loop part is rotated when the wire advances and retracts may form a helical contact protrusion guide part.

Also, the rotating unit may include a contact protrusion configured to protrude in a radial direction from an outer circumferential surface of a wire adjacent to the loop part. A helical groove may be formed so that the contact protrusion comes into contact with an inner portion of the tube and the loop part is rotated when the wire advances and retracts.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a perspective view illustrating a surgical snare instrument according to a first embodiment of the present disclosure;

FIG. 2 is an exploded perspective view of the surgical snare instrument of FIG. 1;

FIGS. 3 to 6 are views illustrating embodiments of a rotating unit of the surgical snare instrument according to the present disclosure; and

FIG. 7 is a perspective view illustrating how the surgical snare instrument is used according to the present disclosure.

### DETAILED DESCRIPTION OF EXEMPLARY EMBODIMENTS

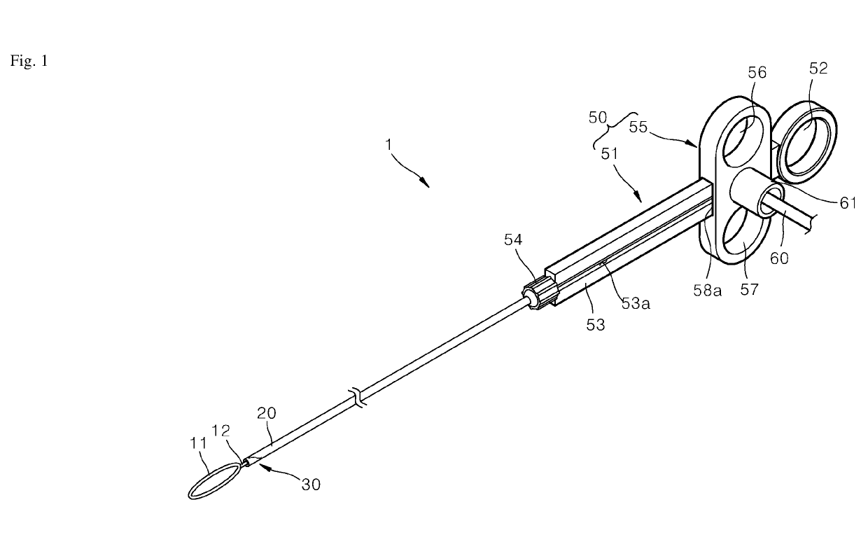

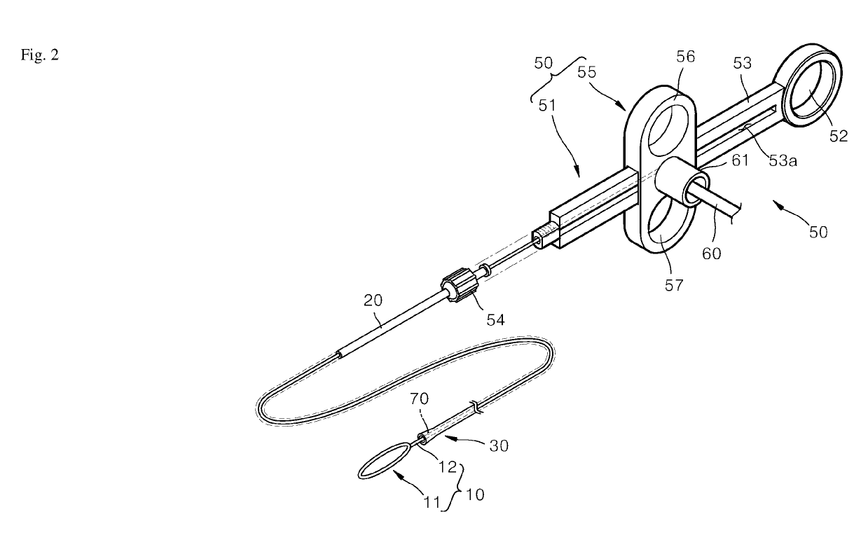

A surgical snare instrument according to the present disclosure is for removing cells, polyps, or the like inside a body, including an organ. An embodiment of a surgical snare instrument is illustrated in FIGS. 1 to 7. Excising intestinal polyps will be described herein as an example.

Referring to the drawings, a surgical snare instrument 1 according to the present disclosure includes a tube 20 having a hollow 21, and an excising unit 10 having a wire 12 installed to be slidable along the hollow 21 of the tube 20 and a loop part 11 installed at an end of the wire 12 and inserted into one end of the tube 20 to be tightened to excise a polyp. Also, the surgical snare instrument 1 includes an advancing-and-retracting unit 50 installed at the other end of the tube 20 to make the wire 12 advance or retract along the hollow 21 of the tube 20. Also, the surgical snare instrument 1 includes a rotating unit 30 installed adjacent to the one end of the tube 20 and configured to rotate the loop part 11 when the wire 12 moves with respect to the tube 20.

Each element of the surgical snare instrument according to the present disclosure configure as described above will be described in more detail below.

According to the present disclosure, the excising unit 10 includes the tube 20 having the hollow, the wire 12 installed to be slidable along the hollow 21 of the tube 20, and the loop part 11 installed at an end of the wire 12 to be inserted into the hollow 21 at one end of the tube 20 to be tightened to excise a polyp. The wire 12 is formed of a metal having elasticity, and plastic deformation of the loop part 11 should not occur when the loop part 11 is inserted and withdrawn into and from the hollow 21 of the tube 20. The excising unit, i.e., the loop part 11 and the wire 12, are preferably formed of a conductor so that a current is received through a terminal 60 of the advancing-and-retracting unit that will be described below and a polyp is excised using a high-frequency current.

The tube 20 may be formed of a synthetic resin having ductility so that the tube 20 is easily movable along a nonlinear insertion path inside a body. The tube 20 is preferably manufactured using a material that does not have much flexibility.

The advancing-and-retracting unit 50 is installed at a side corresponding to a side at which the loop part is exposed through the tube 20 to make the wire 12 advance and retract along the hollow 21 of the tube 20 to enable the wire 12 to be inserted and withdrawn into and from the hollow 21 of the tube 20 with the loop part 11. The advancing-and-retracting unit 50 includes a body portion 51 coupled to an end of the tube 20 and a handle portion 55 to which an end of the wire 12 is connected and configured to move along the body portion 51.

The body portion 51 includes a first insertion portion 52 into which a finger is insertable, an extension frame 53 configured to extend from the first insertion portion 52 and have a guide part 53a formed in the longitudinal direction, and a coupling cover 54 configured to couple an end of the extension frame 53 to the tube 20.

The first insertion portion 52 may be formed in a ring shape so that a finger may be inserted thereinto, and an operator may move the body portion 51 forward and backward with respect to the handle portion 55 while his or her finger is inserted into the first insertion portion 52. Also, the guide part 53a formed in the longitudinal direction in the extension frame 53 is a pathway formed by being inserted into the extension frame 53 in the longitudinal direction, through which the wire 12 of the excising unit 10 moves.

The handle portion 55 is coupled to the extension frame 53 to be movable in the longitudinal direction of the extension frame 53. The handle portion 55 includes a second insertion portion 56 and a third insertion portion 57 into which a finger is insertable, a moving frame 58 configured to connect the second insertion portion 56 and the third insertion portion 57 to each other and have an insertion hole 58a into which the extension frame 53 is inserted, the terminal 60 installed at the moving frame 58 to supply a current to the loop part 11 of the excising unit, and a terminal case 61 configured to surround an outer circumferential surface of the terminal 60. The terminal 60 may be connected to an end of the wire 12 and supply electricity to the loop part 11 of the excising unit.

The second insertion portion 56 and the third insertion portion 57 may be formed in a ring shape so that a finger may be inserted thereinto, and an operator may move the moving frame 58 forward and backward while his or her finger is inserted into the second insertion portion 56 and the third insertion portion 57.

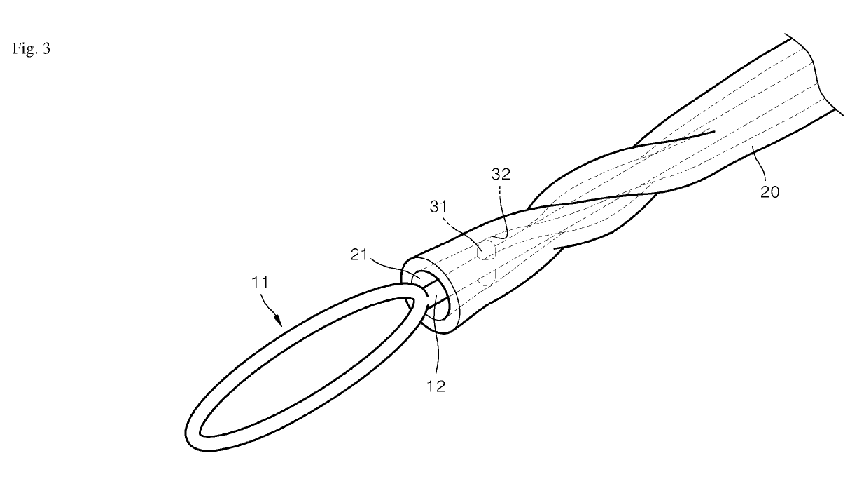

The rotating unit 30 is formed at the wire 12 adjacent to an end of the tube 20 and the loop part to rotate the loop part 11 with respect to the end of the tube 20 to enable an intestinal polyp to be easily inserted into the loop part 11 in a direction in which the intestinal polyp is formed. An embodiment of the rotating unit 30 is illustrated in FIG. 3.

Referring to FIG. 3, the rotating unit 30 includes a contact protrusion 31 configured to protrude in a radial direction from the wire 12 adjacent to the loop part 11. The hollow formed in the tube by an end of the tube 20 helically twisted so that the contact protrusion 31 is guided and the loop part 11 is rotated when the wire 12 advances and retracts may form a helical contact protrusion guide part 32. The contact protrusion guide part 32 may be formed to have a non-circular cross-section. Particularly, an end of the tube 20 for forming the contact protrusion guide part 32 is pressed from both sides so that the hollow of the tube 20 is crushed into a non-circular shape to have a helically twisted structure.

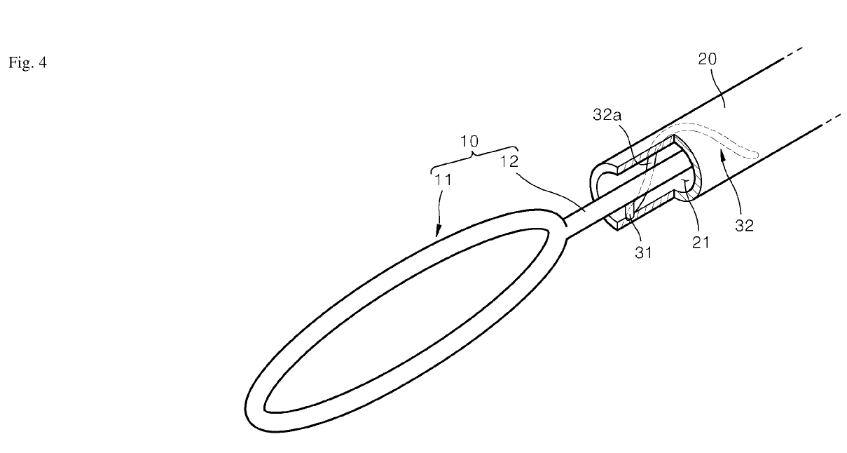

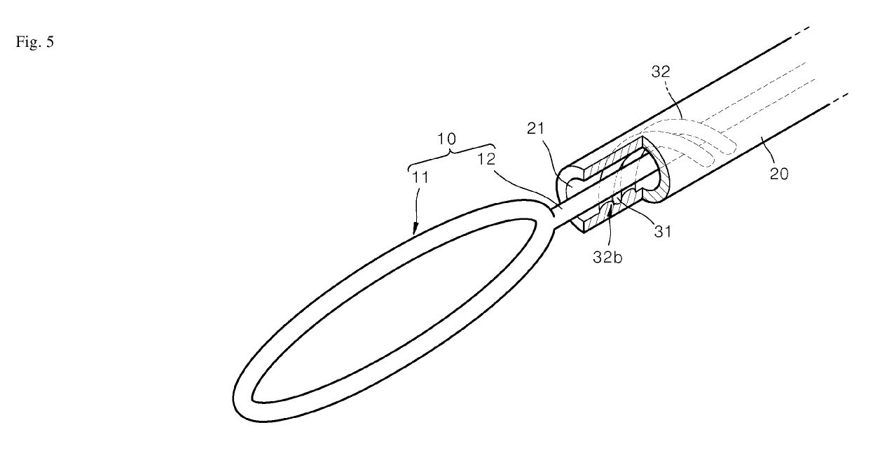

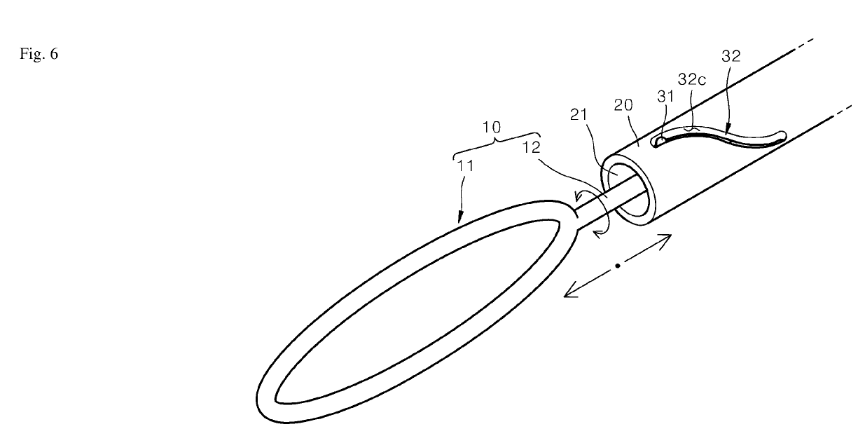

The contact protrusion guide part 32 may include a helical groove 32a formed at an inner circumferential surface of an end of the tube 20 as illustrated in FIG. 4 so that the contact protrusion 31 protruding in the radial direction from the outer circumferential surface of the wire 12 adjacent to the loop part 11 is guided and the loop part 11 is rotated. However, embodiments are not limited thereto, and the contact protrusion guide part 32 may include a helical protrusion 32b configured to guide the contact protrusion 31 to the inner circumferential surface of the end of the tube 20 as illustrated in FIG. 5, or may include a helical through-hole 32c formed at a side of the contact protrusion guide part 32 adjacent to the loop part 11 as illustrated in FIG. 6.

Operation of the surgical snare instrument according to the present disclosure configured as described above will be described below.

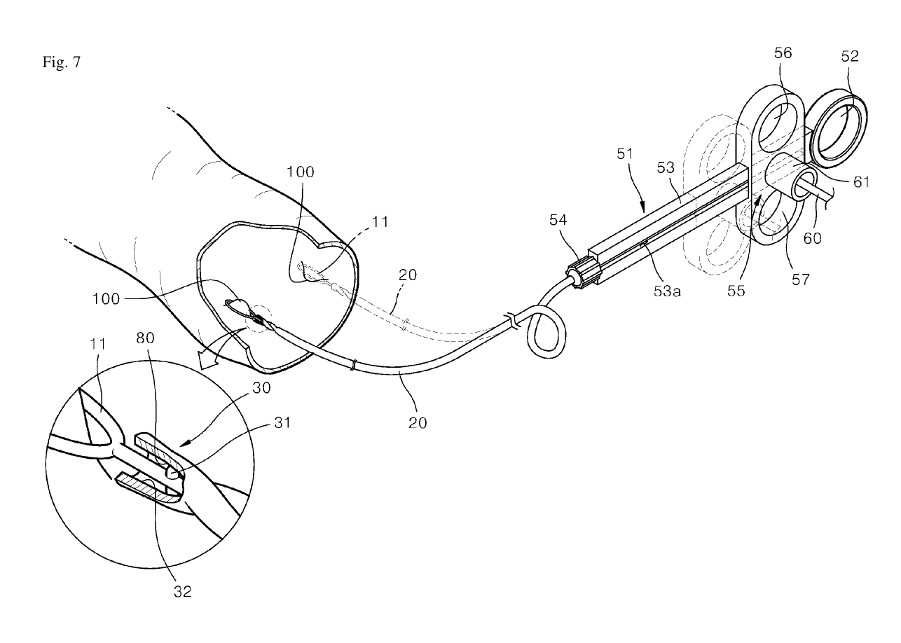

As illustrated in FIG. 7, the surgical snare instrument 1 according to the present disclosure enables an operator to insert his or her fingers into the first insertion portion 52, the second insertion portion 56, and the third insertion portion 57, insert the tube 20 inside a body (inside an intestine), and have the loop part 11 of the excising unit 10 approach a polyp 100 to be excised. Also, the operator widely spreads his or her fingers so that the first insertion portion 52, the second insertion portion 56, and the third insertion portion 57 of the advancing-and-retracting unit 50 are spaced apart from one another and moves the body portion 51 and the handle portion 55 relative to each other, e.g., moves the body portion 51 forward and backward with respect to the handle portion 55, to rotate the loop part 11 so that the polyp is smoothly inserted thereinto.

The rotation of the loop part 11 is performed by the contact protrusion 31, protruding in the radial direction from the wire 12, moving along the helical contact protrusion guide part 32 formed at an end of the tube 20 as illustrated in FIGS. 3 to 7.

The movement of the contact protrusion 31 along the contact protrusion guide part 32 is performed by relatively moving the wire 12 with respect to the tube 20 by the advancing-and-retracting unit 50 as described above.

When the polyp 100 is inserted into the loop part 11 by the actions above, the wire 12 is retracted with respect to the tube 20 to enable the loop part 11 to be inserted into the tube to excise the polyp.

As described above, by the surgical snare instrument 1 according to the present disclosure, the loop part 11 can be rotated to correspond to a position of the polyp 100 by the rotating unit 30 formed at the tube 20 and the wire 12. Thus, an operator can easily perform excision of the polyps without twisting his or her finger in a protruding direction of the polyps.

A surgical snare instrument according to the present disclosure includes a rotating unit and can rotate a loop part so that a loop part of an excising unit is insertable into a part from which a polyp is protruding. In this way, efficiency of surgery for removing polyps can be maximized.

The present disclosure has been described with reference to the embodiments illustrated in the drawings. However, the embodiments are merely illustrative, and one of ordinary skill in the art should understand that the embodiments may be modified in various ways and other equivalent embodiments are possible. Consequently, the actual technical scope of the present disclosure should be defined by the technical spirit of the appended claims.

What is claimed is:

1. A surgical snare instrument comprising:
a tube having a hollow;
an excising unit having a wire installed to be slidable along the hollow of the tube and a loop part installed at an end of the wire and inserted into one end of the tube to be tightened to excise a polyp;
an advancing-and-retracting unit installed at the other end of the tube to make the wire advance or retract along the hollow of the tube; and
a rotating unit installed adjacent to the one end of the tube and configured to rotate the loop part when the wire moves with respect to the tube.

2. The surgical snare instrument of claim 1, wherein the advancing-and-retracting unit includes:
a body portion including a first insertion portion into which a finger is insertable and an extension frame configured to extend from the first insertion portion and have a guide part formed in a longitudinal direction and an end to which the tube is coupled; and
a handle portion including second and third insertion portions into which a finger is insertable and a moving frame configured to connect the second and third insertion portions to each other, have an insertion hole into which the extension frame is inserted to be movable in the longitudinal direction of the extension frame, and have the wire of the excising unit fixed thereto.

3. The surgical snare instrument of claim 1, wherein the rotating unit includes a contact protrusion configured to protrude in a radial direction from a wire adjacent to the loop part, and the hollow formed in the tube by an end of the tube helically twisted so that the contact protrusion is guided and the loop part is rotated when the wire advances and retracts forms a helical contact protrusion guide part.

4. The surgical snare instrument of claim 1, wherein the rotating unit includes a contact protrusion configured to protrude in a radial direction from an outer circumferential surface of a wire adjacent to the loop part, and a helical groove is formed so that the contact protrusion comes into contact with an inner portion of the tube and the loop part is rotated when the wire advances and retracts.

5. The surgical snare instrument of claim 1, wherein the rotating unit includes a contact protrusion configured to protrude in a radial direction from an outer circumferential surface of a wire adjacent to the loop part, and a helical protrusion configured to protrude in a helical shape is formed so that the contact protrusion comes into contact with an inner portion of the tube and the loop part is rotated when the wire advances and retracts.

6. The surgical snare instrument of claim 1, wherein the rotating unit includes a contact protrusion configured to protrude in a radial direction from an outer circumferential surface of a wire adjacent to the loop part, and a helical through-hole is formed at an end of the tube so that the contact protrusion comes into contact and the loop part is rotated when the wire advances and retracts.
